(19) United States
(12) Patent Application Publication
(10) Pub. No.: US 2007/0166416 A1
(43) Pub. Date: Jul. 19, 2007

(54) Formulations and Methods for Preventing and Treating Substance Abuse and Addiction

(76) Inventors: Afa Kehaati Palu, Orem, UT (US); Bing-Nan Zhou, Pleasant Grove, UT (US); Brett Justin West, Orem, UT (US); Claude Jarakae Jensen, Cedar Hills, UT (US); Stephen Story, Alpine, UT (US)

Correspondence Address: KIRTON AND MCCONKIE, 60 EAST SOUTH TEMPLE,, SUITE 1800, SALT LAKE CITY, UT, 84111, US

(21) Appl. No.: 11/686,451

(22) Filed: Mar. 15, 2007

Related U.S. Application Data
(62) Division of application No. 11/076,992, filed on Mar. 9, 2005.

(51) Int. Cl. A61K 36/746; A61K 31/7048; A61K 31/56; A61K 31/375

(52) U.S. Cl. 424/774; 424/777; 514/27; 514/456; 514/725; 514/561; 514/171; 514/474; 514/680; 514/25

(57) ABSTRACT

The invention relates to formulations and methods for preventing and/or treating substance abuse, addiction, withdrawal symptoms, and anticipation associated with substance abuse. In particular the invention relates to formulations comprising at least one processed Morinda citrifolia L. product, and methods comprising the administration of at least one processed Morinda citrifolia L product to prevent and/or treat substance abuse, addiction, withdrawal symptoms, and anticipation associated with substance abuse in living organisms. Embodiments of the invention further comprise Morinda citrifolia based nutraceuticals and administration thereof to affect regulation of dopamine levels, inhibition of opioid receptors, inhibition of mu opioid receptors, inhibition of kappa opioid receptors, inhibition of delta opioid receptors, acting as NMDA antagonist, inhibition of sensitization of NMDA receptors, and/or acting as opiate antagonists.

### BACKGROUND OF THE INVENTION

1. Field of the Invention

The present invention relates to formulations and methods for preventing and treating substance abuse, addiction, withdrawal symptoms, and anticipation associated with substance abuse. In particular the invention relates to formulations comprising at least one processed Morinda citrifolia L. product, and methods comprising the administration of at least one processed Morinda citrifolia L product to prevent and treat substance abuse, addiction, withdrawal symptoms, and anticipation associated with substance abuse in living organisms.

2. Background and Related Art

Most drugs of misuse, except benzodiazepines increase dopamine levels in the mesolimbic dopaminergic system. Consequently, there is significant interest in the mesolimbic system and its relationship to substance abuse. Increased levels of dopamine in the nucleus accumbens are key in mediating the rewarding effects or positive reinforcement of drugs of misuse. Studies have been conducted with humans which provide insight into neurobiological explanations for why drug use is pleasurable and likely to be repeated for some people and unpleasant and not repeated for others. Neuroimaging studies have demonstrated that humans using cocaine or methylphenidate experienced increased dopamine levels in the brain associated with euphoria and pleasure. Low levels of dopamine D2 receptors were associated with pleasure after methylphenidate in drug-naïve individuals, whereas high receptor levels were associated with unpleasant feelings.

Dopamine levels may play an important role in addiction. In particular dopamine levels may play a critical role in anticipation and withdrawl symptoms associated with substance abuse. This insight has been substantiated by experiments which demonstrate that in mammals trained to associate a cue with a pleasurable experience there is an increase in dopaminergic activity in response to the cue and not to the pleasurable experience. If the pleasurable experience was not then presented, dopaminergic function dropped. Reduced dopaminergic function is thought to be associated with negative affect (e.g. dysphoria). Consequently, when an individual with an addiction encounters a ‘cue’ and if their substance of choice is not available may feel dysphoric, which is likely to increase the drive to obtain the substance.

Reduced dopaminergic function has been seen in withdrawal and early abstinence from many drugs of misuse. Neuroimaging studies in cocaine, opiate and alcohol addictions have revealed reduced levels of dopamine D2 receptors, which may recover to some extent during abstinence, but have been shown to persist for months. Early stages of abstinence are associated with elevated levels of craving, drug-seeking and risk of relapse, and it is likely that hypodopaminergic function plays a mediating role. It is possible that the release of dopamine produced by the drug of choice provides relief from withdrawal.

Because of the role of the dopaminergic reward system in addiction, this has been a target for pharmacotherapy. However, results have been mixed. One strategy has been the development of dopaminergic partial agonists at the D3 receptor. Partial agonist stimulate the D3 receptor enough to keep withdrawal at bay, but not enough to cause a ‘high’ or to be rewarding.

An understanding of other neurotransmitter systems that are involved in reward and the modulation of dopaminergic activity provide further targets for pharmacotherapy. The opioid system has three receptor subtypes: mu, kappa and delta. The mu subtype appears to be key in opiate addiction. Mice lacking this receptor did not find morphine rewarding or reinforcing. Further, morphine withdrawal syndrome were not experience by these animals. Additional neuroimaging studies suggest that alterations in mu opiate receptor levels may be fundamental to addiction. Neuroimangeing studies tend to indicate that craving may result from elevated mu opiate receptor levels or decreased endogenous opioid levels.

Roles for kappa and delta opiate receptors in addiction have also been demonstrated experimentally. Unlike mu receptors, kappa receptor stimulation reduces dopamine function in the nucleus accumbens. This may possibly result in dysphoria. In animal models, delta antagonists can reduce self-administration of alcohol, suggesting that this receptor also plays a key role in reinforcement.

As described above, the mu opiate receptor plays a key role in opiate reward, but many of the mechanisms underlying opiate tolerance, dependence and withdrawal remain elusive. As the opiate receptor may not change with chronic opiate exposure, changes ‘downstream’ of the receptor may be more critical. In the treatment of opiate addiction, methadone is the most commonly prescribed drug, although the use of buprenorphine is increasing. Methadone is a full agonist at the mu receptor, whereas buprenorphine is a mu partial agonist. Partial agonists give lower levels of response at maximal receptor occupancy. Also, when a partial agonist occupies receptors, fewer are available for a full agonist (e.g. heroin). The partial agonist is therefore acting as an antagonist. Consequently, buprenorphine will stimulate the mu opioid receptor, but not maximally (hence, there is less risk of respiratory depression in overdose), and will also prevent the effects of heroin taken ‘on top’. In addition, its longer half-life allows less than daily dosing, an advantage in supervised consumption.

Ecstasy (3,4-methylenedioxymethamphetamine or MDMA) and its derivatives MDA and MDEA have both stimulant and hallucinogenic properties. Acutely, MDMA increases 5-hydroxytryptamine (5-HT or serotonin) levels, and, to a lesser extent, dopamine levels, by stimulating release and inhibiting uptake. Animal studies have revealed ecstasy and its derivatives to be neurotoxic to serotonergic neurons (MDA>MDMA>MDEA), but it is controversial whether and to what extent the same occurs in man (Boot et al, 2000). Neuroimaging studies using PET and single photon emission tomography (SPET) to measure 5-HT transporter levels in persons who are regular heavy ecstasy users report reduced levels. However, methodological questions about the tracer, contribution of blood flow and choice of subjects necessarily limit these conclusions (Semple et al, 1999; Reneman et al, 2001). There is some evidence for cognitive impairments in individuals using ecstasy which may persist after a period of chronic use, and it is not clear how reversible these are with time. In animal models, fluoxetine has been shown to be neuroprotective, apparently by blocking ecstasy uptake into 5-HT neurons, but it is unknown whether this protective effect occurs in humans.

### SUMMARY OF THE INVENTION

The invention relates to formulations and methods for preventing and/or treating substance abuse, addiction, withdrawal symptoms, and anticipation associated with substance abuse. In particular the invention relates to formulations comprising at least one processed Morinda citrifolia L. product, and methods comprising the administration of at least one processed Morinda citrifolia L product to prevent and/or treat substance abuse, addiction, withdrawal symptoms, and anticipation associated with substance abuse in living organisms. Embodiments of the invention further comprise Morinda citrifolia based nutraceuticals and administration thereof to affect regulation of dopamine levels, inhibition of opioid receptors, inhibition of mu opioid receptors, inhibition of kappa opioid receptors, inhibition of delta opioid receptors, acting as NMDA antagonist, inhibition of sensitization of NMDA receptors, and/or acting as opiate antagonists.

Methods of the present invention also comprise the obtaining of Morinda citrifolia compositions and extracts, including Morinda citrifolia fruit juice and concentrates thereof.

The formulations of the invention comprise processed Morinda citrifolia products. In one embodiment, the formulations include one or more extracts from the Morinda citrifolia L. plant. The Morinda citrifolia extracts preferably include Morinda citrifolia fruit juice, which juice is preferably present in an amount capable of maximizing prevention and/or treatment of substance abuse, addiction, withdrawal symptoms, anticipation associated with substance abuse in living organisms and associated symptoms. In another preferred embodiment the processed Morinda citrifolia product is administer in an amount sufficient to regulate dopamine levels, inhibit opioid receptors, inhibit mu opioid receptors, inhibit kappa opioid receptors, inhibit delta opioid receptors, act as NMDA antagonist, inhibit sensitization of NMDA receptors, and/or act as opiate antagonists without causing negative side effects when the composition is administered to a mammal.

These and other features and advantages of the present invention will be set forth or will become more fully apparent in the description that follows and in the appended claims. The features and advantages may be realized and obtained by means of the instruments and combinations particularly pointed out in the appended claims. Furthermore, the features and advantages of the invention may be learned by the practice of the invention or will be obvious from the description, as set forth hereinafter.

### DETAILED DESCRIPTION OF THE INVENTION

The following description of embodiments of the methods and compositions of the present invention is not intended to limit the scope of the invention, but is merely representative of some embodiments, including the preferred embodiments, of the present invention.

The invention relates to formulations and methods for preventing and/or treating substance abuse, addiction, withdrawal symptoms, and anticipation associated with substance abuse. In particular the invention relates to formulations comprising at least one processed Morinda citrifolia L. product, and methods comprising the administration of at least one processed Morinda citrifolia L product to prevent and/or treat substance abuse, addiction, withdrawal symptoms, and anticipation associated with substance abuse in living organisms. Embodiments of the invention further comprise Morinda citrifolia based nutraceuticals and administration thereof to affect regulation of dopamine levels, inhibition of opioid receptors, inhibition of mu opioid receptors, inhibition of kappa opioid receptors, inhibition of delta opioid receptors, acting as NMDA antagonist, inhibition of sensitization of NMDA receptors, and/or acting as opiate antagonists.

The present invention comprises Morinda citrifolia compositions, each of which is comprised of one or more processed Morinda citrifolia L. products. The processed Morinda citrifolia product(s) preferably includes Morinda citrifolia fruit juice, which juice is preferably present in an amount capable of preventing and treating substance abuse and addiction without causing negative side effects when the composition is administered to a mammal. The composition comprising Morinda citrifolia may be comprised of extracts from the Morinda citrifolia L. plant. The extracts may be selected from a list comprising: fruit, fruit juice, fruit pulp, fruit juice concentrates, fruit pulp concentrates, fruit puree, fruit juice in combination with fruit pulp, leaves, leaf extracts, seeds, seed extracts, flowers, roots, bark, and wood.

Some compositions of the present invention comprise Morinda citrifolia extracts present between about 1 and 5 percent of the weight of the total composition. Other such percentage ranges include: about 0.1 and 50 percent; about 85 and 99 percent; about 5 and 10 percent; about 10 and 15 percent; about 15 and 20 percent; about 20 and 50 percent; and about 50 and 100 percent.

In some Morinda citrifolia compositions of the present invention, Morinda citrifolia fruit juice evaporative concentrate is present, the evaporative concentrate having a concentration strength (described further herein) between about 8 and 12 percent.

In some Morinda citrifolia compositions of the present invention, Morinda citrifolia fruit juice freeze concentrate is present, the freeze concentrate having a concentration strength (described further herein) between about 8 and 12 percent. Other such percentage ranges include: about 4 and 12 percent; and about 0.5 and 12 percent.

One or more Morinda citrifolia extracts can be further combined with other ingredients or carriers (discussed further herein) to produce a pharmaceutical Morinda citrifolia product or composition (“pharmaceutical” herein referring to any drug or product designed to improve the health of living organisms such as human beings or mammals, including nutraceutical products) that is also a Morinda citrifolia of the present invention. Examples of pharmaceutical Morinda citrifolia products may include, but are not limited to, orally administered solutions and intravenous solutions.

Methods of the present invention comprise the administration of Morinda citrifolia compositions in amounts to prevent and/or treat substance abuse, addiction, withdrawal symptoms, and anticipation associated with substance abuse. Embodiments of the invention further comprise methods for administering Morinda citrifolia based nutraceuticals and to affect regulation of dopamine levels, inhibition of opioid receptors, inhibition of mu opioid receptors, inhibition of kappa opioid receptors, inhibition of delta opioid receptors, acting as NMDA antagonist, inhibition of sensitization of NMDA receptors, and/or acting as opiate antagonists. It will be understood that specific dosage levels of any compositions that will be administered to any particular patient will depend upon a variety of factors, including the patient's age, body weight, general health, gender, diet, time of administration, route of administration, rate of excretion, and drug combination(s).

Methods of the present invention also include the obtaining of Morinda citrifolia compositions and extracts, including Morinda citrifolia fruit juice and concentrates thereof. It will be noted that some of the embodiments of the present invention contemplate obtaining the Morinda citrifolia fruit juice pre-made. Various methods of the present invention shall be described in more detail further herein.

The following disclosure of the present invention is grouped into subheadings, The utilization of the subheadings is for convenience of the reader only and is not to be construed as limiting in any sense.

1. Obtaining Extracts from Morinda citrifolia Plant for Incorporation into the Compositions of the Present Invention

The Indian Mulberry or Noni plant, known scientifically as Morinda citrifolia L. (Morinda citrifolia), is a shrub or small tree. The leaves are oppositely arranged with an elliptic to ovate form. The small white flowers are contained in a fleshy, globose, head-like cluster. The fruits are large, fleshy, and ovoid. At maturity, they are creamy-white and edible, but have an unpleasant taste and odor. The plant is native to Southeast Asia and has spread in early times to a vast area from India to eastern Polynesia. It grows randomly in the wild, and it has been cultivated in plantations and small individual growing plots. The Morinda citrifolia flowers are small, white, three to five lobed, tubular, fragrant, and about 1.25 cm long. The flowers develop into compound fruits composed of many small drupes fused into an ovoid, ellipsoid or round, lumpy body, with waxy, white, or greenish-white or yellowish, semi-translucent skin. The fruit contains “eyes” on its surface, similar to a potato. The fruit is juicy, bitter, dull-yellow or yellowish-white, and contains numerous red-brown, hard, oblong-triangular, winged 2-celled stones, each containing four seeds.

When fully ripe, the fruit has a pronounced odor like rancid cheese. Although the fruit has been eaten by several nationalities as food, the most common use of the Morinda citrifolia plant was as a red and yellow dye source. Recently, there has been an interest in the nutritional and health benefits of the Morinda citrifolia plant, further discussed below.

Processed Morinda citrifolia fruit juice can be prepared by separating seeds and peels from the juice and pulp of a ripened Morinda citrifolia fruit; filtering the pulp from the juice; and packaging the juice. Alternatively, rather than packaging the juice, the juice can be immediately included as an ingredient in other products. In some embodiments, the juice and pulp can be pureed into a homogenous blend to be mixed with other ingredients. Other processes include freeze-drying the fruit and juice. The fruit and juice can be reconstituted during production of the final juice product. Still other processes include air-drying the fruit and juices, prior to being masticated.

The present invention also contemplates the use of fruit juice and/or puree fruit juice extracted from the Morinda citrifolia plant. In a currently preferred process of producing Morinda citrifolia fruit juice, the fruit is either hand picked or picked by mechanical equipment. The fruit can be harvested when it is at least one inch (2-3 cm) and up to 12 inches (24-36 cm) in diameter. The fruit preferably has a color ranging from a dark green through a yellow-green up to a white color, and gradations of color in between. The fruit is thoroughly cleaned after harvesting and before any processing, occurs.

The fruit is allowed to ripen or age from 0 to 14 days, with most fruit being held from 2 to 3 days. The fruit is ripened or aged by being placed on equipment so it does not contact the ground. It is preferably covered with a cloth or netting material during aging, but can be aged without being covered. When ready for further processing the fruit is light in color, from a light green, light yellow, white or translucent color. The fruit is inspected for spoilage or for excessively green color and hard firmness. Spoiled and hard green fruit is separated from the acceptable fruit.

The ripened and aged fruit may be placed in containers for processing and transport. In a preferred embodiment of the invention, the aged fruit is placed in plastic lined containers for further processing and transport. The containers of aged fruit may be held from 0 to 120 days. In a preferred embodiment of the invention, the fruit containers are held for 7 to 14 days before processing. The containers can optionally be stored under refrigerated conditions or ambient/room temperature conditions prior to further processing. The fruit is unpacked from the storage containers and may be further processed through a manual or mechanical separator, in which the seeds and peel are separated from the juice and pulp.

The juice and pulp can be packaged into containers for storage and transport. Alternatively, the juice and pulp can be immediately processed into a finished juice product. The containers can be stored in refrigerated, frozen, or room temperature conditions.

The Morinda citrifolia juice and pulp are preferably blended in a homogenous blend, after which they may be mixed with other ingredients. The finished juice product is preferably heated and pasteurized at a minimum temperature of 181° F. (83° C.) or higher up to 212° F. (100° C.).

Another product manufactured is Morinda citrifolia puree and puree juice, in either concentrate or diluted form. Puree is essentially the pulp separated from the seeds and is different from the fruit juice product described herein.

Each product is filled and sealed into a final container. The container may be plastic, glass, or another suitable material that can withstand the processing temperatures. The containers are maintained at the filling temperature or may be cooled rapidly and then placed in a shipping container. The shipping containers are preferably wrapped with a material and in a manner to maintain or control the temperature of the product in the final containers.

The juice and pulp may be further processed by separating the pulp from the juice through filtering equipment. The filtering equipment preferably consists of, but is not limited to, a centrifuge decanter, a screen filter with a size from 0.01 micron up to 2000 microns, more preferably less than 500 microns, a filter press, reverse osmosis filtration, and any other standard commercial filtration devices. The operating filter pressure preferably ranges from 0.1 psig up to about 1000 psig. The flow rate preferably ranges from 0.1 g.p.m. up to 1000 g.p.m., and more preferably between 5 and 50 g.p.m. The wet pulp may be washed and filtered at least once and up to 10 times to remove any juice from the pulp. The wet pulp typically has a fiber content of 10 to 40 percent by weight. The wet pulp is preferably pasteurized at a temperature of 181° F. (83° C.) minimum and then packed in drums for further processing or made into a high fiber product.

The processed Morinda citrifolia product may also exist as a fiber. Still further, the processed Morinda citrifolia product may also exist in oil form, such as an oil extract. The Morinda citrifolia oil typically includes a mixture of several different fatty acids as triglycerides, such as palmitic, stearic, oleic, and linoleic fatty acids, and other fatty acids present in lesser quantities. In addition, the oil preferably includes an antioxidant to inhibit spoilage of the oil. Conventional food grade antioxidants are preferably used.

The high fiber product may include wet or dry Morinda citrifolia pulp, supplemental fiber ingredients, water, sweeteners, flavoring agents, coloring agents, and/or nutritional ingredients. The supplemental fiber ingredients may include plant based fiber products, either commercially available or developed privately. Examples of some typical fiber products are guar gum, gum arabic, soybean fiber, oat fiber, pea fiber, fig fiber, citrus pulp sacs, hydroxymethylcellulose, cellulose, seaweed, food grade lumber or wood pulp, hemicellulose, etc. Other supplemental fiber ingredients may be derived from grains or grain products. The concentrations of these other fiber raw materials typically range from 0 up to 30 percent, by weight, and more preferably from 10 to 30 percent by weight.

The juice and pulp can be dried using a variety of methods. The juice and pulp mixture can be pasteurized or enzymatically treated prior to drying. The enzymatic process begins with heating the product to a temperature between 75° F. and 135° F. It is then treated with either a single enzyme or a combination of enzymes. These enzymes include, but are not limited to, amylase, lipase, protease, cellulase, bromelin, etc. The juice and pulp may also be dried with other ingredients, such as those described above in connection with the high fiber product. The typical nutritional profile of the dried juice and pulp is 1 to 20 percent moisture, 0.1 to 15 percent protein, 0.1 to 20 percent fiber, and the vitamin and mineral content.

The filtered juice and the water from washing the wet pulp are preferably mixed together. The filtered juice may be vacuum evaporated to a brix of 40 to 70 and a moisture of 0.1 to 80 percent, more preferably from 25 to 75 percent. The resulting concentrated Morinda citrifolia juice may or may not be pasteurized. For example, the juice would not be pasteurized in circumstances where the sugar content or water activity was sufficiently low enough to prevent microbial growth.

The Morinda citrifolia plant is rich in natural ingredients. Those ingredients that have been discovered include: (from the leaves): alanine, anthraquinones, arginine, ascorbic acid, aspartic acid, calcium, beta-carotene, cysteine, cystine, glycine, glutamic acid, glycosides, histidine, iron, leucine, isoleucine, methionine, niacin, phenylalanine, phosphorus, proline, resins, riboflavin, serine, beta-sitosterol, thiamine, threonine, tryptophan, tyrosine, ursolic acid, and valine; (from the flowers): acacetin-7-o-beta-d(+)-glucopyranoside, 5,7-dimethyl-apigenin-4′-o-beta-d(+)-galactopyranoside, and 6,8-dimethoxy-3-methylanthraquinone-1-o-beta-rhamnosyl-glucopyranoside; (from the fruit): acetic acid, asperuloside, butanoic acid, benzoic acid, benzyl alcohol, 1-butanol, caprylic acid, decanoic acid, (E)-6-dodeceno-gamma-lactone, (Z,Z,Z)-8,11,14-eicosatrienoic acid, elaidic acid, ethyl decanoate, ethyl hexanoate, ethyl octanoate, ethyl palmitate, (Z)-6-(ethylthiomethyl) benzene, eugenol, glucose, heptanoic acid, 2-heptanone, hexanal, hexanamide, hexanedioic acid, hexanoic acid (hexoic acid), 1-hexanol, 3-hydroxy-2-butanone, lauric acid, limonene, linoleic acid, 2-methylbutanoic acid, 3-methyl-2-buten-1-ol, 3-methyl-3-buten-1-ol, methyl decanoate, methyl elaidate, methyl hexanoate, methyl 3-methylthio-propanoate, methyl octanoate, methyl oleate, methyl palmitate, 2-methylpropanoic acid, 3-methylthiopropanoic acid, myristic acid, nonanoic acid, octanoic acid (octoic acid), oleic acid, palmitic acid, potassium, scopoletin, undecanoic acid, (Z,Z)-2,5-undecadien-1-ol, and vomifol; (from the roots): anthraquinones, asperuloside (rubichloric acid), damnacanthal, glycosides, morindadiol, morindine, morindone, mucilaginous matter, nor-damnacanthal, rubiadin, rubiadin monomethyl ether, resins, soranjidiol, sterols, and trihydroxymethyl anthraquinone-monomethyl ether; (from the root bark): alizarin, chlororubin, glycosides (pentose, hexose), morindadiol, morindanigrine, morindine, morindone, resinous matter, rubiadin monomethyl ether, and soranjidiol; (from the wood): anthragallol-2,3-dimethylether; (from the tissue culture): damnacanthal, lucidin, lucidin-3-primeveroside, and morindone-6beta-primeveroside; (from the plant): alizarin, alizarin-alpha-methyl ether, anthraquinones, asperuloside, hexanoic acid, morindadiol, morindone, morindogenin, octanoic acid, and ursolic acid.

The present invention contemplates utilizing all parts of the M. citrifolia plant alone, in combination with each other or in combination with other ingredients. The above listed portions of the M. citrifolia plant are not an exhaustive list of parts of the plant to be used but are merely exemplary. Thus, while some of the parts of the M. citrifolia plant are not mentioned above (e.g., seed from the fruit, the pericarp of the fruit, the bark or the plant) the present invention contemplates the use of all of the parts of the plant.

Ingredients, components or extracts may be obtained from any part of the Morinda citrifolia plant including leaves, stem, seeds and/or roots. In a preferred embodiment of the invention, extracts may be obtained from the leaves, stem, seeds, and/or roots by first chopping the raw material. Next, an extraction method may be utilized to isolate ingredients of interest. Extraction of ingredients of interest may be accomplished by exposing the raw ingredients to a solvent of choice. In one embodiment of the invention, a hot water extraction method is utilized, at an appropriate temperature to ensure isolation of the desired ingredients. For example, water may be added to the raw materials in a five to one ratio by weight and heated to 95° C. Other solvents may be utilized for the extraction including organic solvents or mixtures of aqueous and organic solvents. Organic solvents are preferably selected from a list comprising ethanol, methanol, and hexane. Moreover, wet pressure and heat process using ordinary autoclave equipment may be applied. Furthermore, treatment processes using cellulose hydrolysis enzyme may be added to aforementioned processes. After removing insoluble components through filtering, if desired, from extract obtained from leaves, stems, seeds and/or roots, solvent is removed and extract of the present invention is obtained. This extract may be pasteurized, if necessary, or concentrated or dried. Drying may be achieved using ordinary spray drying or freeze-drying. The extract may be stored under cooling or freezing conditions.

Moreover, oil may be extracted from seeds. Oil may be obtained by drying, crushing, and squeezing seeds with a press. More oil may be extracted from seed cake residue by extracting the oil utilizing a solvent selected from a list comprising hexane, ethanol, water, other aqueous solvents, or other organic solvent. The oil contains fatty acid such as linoleic acid, oleic acid, palmitic acid and stearic acid in the form of triglycerides.

Recently, as mentioned, many health benefits have been discovered stemming from the use of products containing Morinda citrifolia. One benefit of Morinda citrifolia is found in its ability to isolate and produce Xeronine. Xeronine occurs in practically all healthy cells of plants, animals and microorganisms. Even though Morinda citrifolia has a negligible amount of free Xeronine, it contains appreciable amounts of the precursor of Xeronine, called Proxeronine. Further, Morinda citrifolia contains the inactive form of the enzyme Proxeronase, which releases Xeronine from Proxeronine. A paper entitled, “The Pharmacologically Active Ingredient of Noni” by R. M. Heinicke of the University of Hawaii, which is incorporated by reference in its entirety, indicates that Morinda citrifolia is “the best raw material to use for the isolation of xeronine,” because of the building blocks of Proxeronine and Proxeronase.

Xeronine protects and keeps the shape and suppleness of protein molecules so that they may be able to pass through the cell walls and be used to form healthy tissue. Without these nutrients going into the cell, the cell cannot perform its job efficiently. Xeronine assists in enlarging the membrane pores of the cells. This enlargement allows for larger chains of peptides (amino acids or proteins) to be admitted into the cell. If these chains are not used, they become waste. Additionally, Xeronine, which is made from Proxeronine, assists in enlarging the pores to allow better absorption of nutrients. Because of its many benefits, Morinda citrifolia has been known to provide a number of anecdotal effects

As used herein, the term Morinda citrifolia juice refers to a product that includes juice processed from the fruit of the Indian Mulberry or Morinda citrifolia L. plant. In one embodiment, Morinda citrifolia juice includes reconstituted fruit juice from pure juice puree of French Polynesia. The composition or formulation comprising at least one processed Morinda citrifolia product may also include other ingredients. In a further embodiment, Morinda citrifolia juice is not processed from dried or powdered Morinda citrifolia.

2. Formulations and Methods of Administration

The compositions of the present invention may be formulated into any of a variety of compositions, including orally administered compositions, intravenous solutions, and other products or compositions. As mentioned earlier herein, the compositions can include a variety of ingredients.

Orally administered compositions may take the form of, for example, liquids, beverages, tablets, lozenges, aqueous or oily suspensions, dispersible powders or granules, emulsions, syrups, or elixirs. Compositions intended for oral use may be prepared according to any method known in the art, and such compositions may contain one or more agents such as sweetening agents, flavoring agents, coloring agents, and preserving agents. They may also contain one or more additional ingredients such as vitamins and minerals, etc. Tablets may be manufactured to contain one or more Morinda citrifolia extracts in admixture with non-toxic, pharmaceutically acceptable excipients that are suitable for the manufacture of tablets. These excipients may be, for example, inert diluents, granulating and disintegrating agents, binding agents, and lubricating agents. The tablets may be uncoated or they may be coated by known techniques to delay disintegration and absorption in the gastrointestinal tract and thereby provide sustained action over a longer period. For example, a time delay material such as glyceryl monostearate or glyceryl distearate may be used.

Aqueous suspensions may be manufactured to contain Morinda citrifolia extracts in admixture with excipients suitable for the manufacture of aqueous suspensions. Examples of such excipients include, but are not limited to: suspending agents such as sodium carboxymethyl-cellulose, methylcellulose, hydroxy-propylmethycellulose, sodium alginate, polyvinyl-pyrrolidone, gum tragacanth and gum acacia; dispersing or wetting agents such as a naturally-occurring phosphatide like lecithin, or condensation products of an alkylene oxide with fatty acids such as polyoxyethylene stearate, or condensation products of ethylene oxide with long chain aliphatic alcohols such as heptadecaethylene-oxycetanol, or condensation products of ethylene oxide with partial esters derived from fatty acids and a hexitol such as polyoxyethylene sorbitor monooleate, or condensation products of ethylene oxide with partial esters derived from fatty acids and hexitol anhydrides such as polyethylene sorbitan monooleate.

Typical sweeteners may include, but are not limited to, natural sugars derived from corn, sugar beet, sugar cane, potato, tapioca, or other starch-containing sources that can be chemically or enzymatically converted to crystalline chunks, powders, and/or syrups. In addition, sweeteners can consist of artificial or high intensity sweeteners, some of which are aspartame, sucralose, stevia, saccharin, etc. The concentration of sweeteners may be between from 0 to 50 percent by weight, of the formula, and more preferably between about 1 and 5 percent by weight.

Typical flavors can include, but are not limited to, artificial and/or natural flavor or ingredients that contribute to palatability. Natural flavors include but are not limited to other fruits and vegetables. The concentration of flavors may range, for example, from 0 up to 15 percent by weight, of the formula. Colors may include food grade artificial or natural coloring agents having a concentration ranging from 0 up to 10 percent by weight, of the formula.

Typical nutritional ingredients may include vitamins, minerals, trace elements, herbs, botanical extracts, bioactive chemicals and compounds at concentrations from 0 up to 10 percent by weight. Examples of vitamins one can add to the fiber composition include, but are not limited to, vitamins A, B1 through B12, C, D, E, Folic Acid, Pantothenic Acid, Biotin, etc. Examples of minerals and trace elements one can add to the fiber composition include, but are not limited to, calcium, chromium, copper, cobalt, boron, magnesium, iron, selenium, manganese, molybdenum, potassium, iodine, zinc, phosphorus, etc. Herbs and botanical extracts include, but are not limited to, alfalfa grass, bee pollen, chlorella powder, Dong Quai powder, Ecchinacea root, Gingko Biloba extract, Horsetail herb, Indian mulberry, Shitake mushroom, spirulina seaweed, grape seed extract, etc. Typical bioactive chemicals may include, but are not limited to, caffeine, ephedrine, L-carnitine, creatine, lycopene, etc.

Ingredients of the present invention may also include one or more carrier agents (for example, water) known or used in the art. Examples of other ingredients may include, but are not limited to artificial flavoring, other natural juices or juice concentrates such as a natural grape juice concentrate or a natural blueberry juice concentrate. The ingredients to be utilized in the compositions of the present invention may include any that are safe for internalizing into the body of a mammal.

Favorably, this invention provides a method of diabetes with a Morinda citrifolia-based formulation without any significant tendency to cause undesirable side effects.

The present invention features a unique formulation and method of administering the same to prevent and/or treat substance abuse, addiction, withdrawal symptoms and/or anticipation, by providing a nutraceutical composition or treatment formulated with one or more processed Morinda citrifolia products derived from the Indian Mulberry plant. The Morinda citrifolia product is incorporated into various carriers or nutraceutical compositions suitable for in vivo treatment of a patient. For instance, the nutraceutical formulation may be ingested orally, introduced via an intravenous injection or feeding system, or otherwise internalized as is appropriate and directed.

The nutraceutical composition of the present invention comprises one or more of a processed Morinda citrifolia product present in an amount by weight between about 0.01 and 100 percent by weight, and preferably between 0.01 and 95 percent by weight. Several exemplary embodiments of formulations are provided below. However, these are only intended to be exemplary, as one ordinarily skilled in the art will recognize other formulations or compositions comprising the processed Morinda citrifolia product.

The processed Morinda citrifolia product is the active ingredient or contains one or more active ingredients, such as quercetin, rutin, scopoletin, octoanoic acid, potassium, vitamin C, terpenoids, alkaloids, anthraquinones (such as nordamnacanthal, morindone, rubiandin, B-sitosterol, carotene, vitamin A, flavone glycosides, linoleic acid, Alizarin, amino acides, acubin, L-asperuloside, caproic acid, caprylic acid, ursolic acid, and a putative proxeronine and others, for treating and preventing substance abuse, addiction, withdrawal symptoms and/or anticipation. Active ingredients may be extracted utilizing aqueous or organic solvents including various alcohol or alcohol-based solutions, such as methanol, ethanol, and ethyl acetate, and other alcohol-based derivatives using any known process in the art. The active ingredients of quercetin and rutin may be present in amounts by weight ranging from 0.01-10 percent of the total formulation or composition. These amounts may be concentrated as well into a more potent concentration in which they are present in amounts ranging from 10 to 100 percent.

The nutraceutical composition comprising Morinda citrifolia may be prepared using any known means in the art. In addition, since the nutraceutical composition will most likely be consumed orally, it may contain one or more agents selected from the group consisting of sweetening agents, flavoring agents, coloring agents, preserving agents, and other medicinal agents as directed.

The present invention further features formulations and methods of administering said formulations comprising one or more processed Morinda citrifolia products to regulate dopamine levels, inhibit opioid receptors comprising mu, kappa and delta opioid receptors, act as NMDA antagonist, inhibit sensitization of NMDA receptors, and/or act as opiate antagonists by providing a nutraceutical composition or treatment formulated. A preferred embodiment for administering the nutraceuticals of the present invention comprises the steps of (a) formulating a nutraceutical composition comprising a processed Morinda citrifolia product present in an amount between about 0.01 and 95 percent by weight, wherein the composition also comprises a carrier, such as water or purified water, and other natural or artificial ingredients; (b) introducing the nutraceutical composition into the body, such that the processed Morinda citrifolia product is sufficiently internalized; (c) repeating the above steps as often as necessary to provide an effective amount of the processed Morinda citrifolia product to the body of the patient to prevent and/or treat substance abuse, addiction, withdrawal symptoms associated with substance abuse, anticipation associated with substance abuse, regulate dopamine levels, inhibit opioid receptors, inhibit mu opioid receptors, inhibit kappa opioid receptors, inhibit delta opioid receptors, act as NMDA antagonist, inhibit sensitization of NMDA receptors, and/or act as opiate antagonists.

The step of introducing the nutraceutical composition into the body comprises one of ingesting the composition orally. Ingesting the nutraceutical orally means the nutraceutical composition may be formulated as a liquid, gel, solid, or some other type that would allow the composition to be quickly digested and concentrated within the body. It is important to note that the step of administering the nutraceutical composition should be carried out in an effective manner so that the greatest concentration of nutraceutical composition, and particularly the processed Morinda citrifolia product, is internalized and absorbed into the patient's body. In one embodiment, the nutraceutical composition is administered by taking between 1 teaspoon and 2 oz., and preferably 2 oz., of the nutraceutical composition every two hours each day, or at least twice a day. The invention specifically contemplates administering less than one ounce including 0.001 ounces. The invention specifically contemplates administering one ounce, two ounces, three ounces, four ounces, five ounces, six ounces, seven ounces, eight ounces, nine ounces, ten ounces or any fraction of an ounce in between the above specified dosages at each administration of the nutraceutical composition. In addition, the nutraceutical composition may be taken on an empty stomach, meaning at a period of time at least two hours prior to consumption of any food or drink. Following this, the nutraceutical composition is sufficiently allowed to absorb into the tissues of the body. Of course, one ordinarily skilled in the art will recognize that the amount of composition and frequency of use may vary from individual to individual. For example, the invention contemplates the administration of up to 10 ozs. for each administration. The invention specifically contemplates administering a given dosage of the nutraceutical composition once each day, twice each day, three each per day, four times each day, five times each day, six times each day, seven times each day, eight times each day, nine times each day, ten times each day or more depending upon need as determined by indicia described above including the age of patient being treated, weight of patient , severity of symptoms, and desired results.

In another method of the present invention, a person suffering from addiction, withdrawal or anticipation takes at least one (1) ounce of Formulation One in the morning on an empty stomach, and at least one (1) ounce at night on an empty stomach, just prior to retiring to bed. In another method of the present invention, a person diagnosed with or experiencing addiction, withdrawal or anticipation takes at least one ounce of Formulation Two twice a day. In addition, the step of administering the nutraceutical composition may include injecting the composition into the body using an intravenous pump.

The following compositions or formulations represent some of the preferred embodiments contemplated by the present invention.

Formulation One

| Ingredients | Percent by Weight |
|---|---|
| Morinda citrifolia fruit juice | 100% |

Formulation Two

| Ingredients | Percent by Weight |
|---|---|
| Morinda citrifolia fruit juice | 85-99.99% |
| Water | 0.1-15% |

Formulation Three

| Ingredients | Percent by Weight |
|---|---|
| Morinda citrifolia fruit juice | 85-99.99 |
| Other fruit juices | 0.1-15% |

Formulation Four

| Ingredients | Percent by Weight |
|---|---|
| Morinda citrifolia fruit juice | 50-90% |
| Water | 0.1-50% |
| Other fruit juices | 0.1-30% |

Formulation Five

| Ingredients | Percent by Weight |
|---|---|
| Morinda citrfolia extract | 100% |

Formulation Six

| Ingredients | Percent by Weight |
|---|---|
| Morinda citrifolia extract | 50-90% |
| Water | 0.1-50% |

Formulation Seven

| Ingredients | Percent by Weight |
|---|---|
| Morinda citrifolia extract | 50-90% |
| Other fruit juices | 0.1-30% |

Formulation Eight

| Ingredients | Percent by Weight |
|---|---|
| Morinda citrifolia extract | 50-90% |
| Water | 0.1-50% |
| Other fruit juices | 0.1-30% |

Formulation Nine

| Ingredients | Percent by Weight |
|---|---|
| Morinda citrifolia extract | 0.1-50% |
| Water | 0.1-50% |

3. Opiate Receptor Antagonist

The invention relates to formulations and methods for preventing and/or treating substance abuse, addiction, withdrawal symptoms, and anticipation associated with substance abuse. In particular the invention relates to formulations comprising at least one processed Morinda citrifolia L. product, and methods comprising the administration of at least one processed Morinda citrifolia L product to prevent and/or treat substance abuse, addiction, withdrawal symptoms, and anticipation associated with substance abuse in living organisms. Embodiments of the invention further comprise Morinda citrifolia based nutraceuticals and administration thereof to affect regulation of dopamine levels, inhibition of opioid receptors, inhibition of mu opioid receptors, inhibition of kappa opioid receptors, inhibition of delta opioid receptors, acting as NMDA antagonist, inhibition of sensitization of NMDA receptors, and/or acting as opiate antagonists.

Methods of the present invention also comprise the obtaining of Morinda citrifolia compositions and extracts, including Morinda citrifolia fruit juice and concentrates thereof.

The formulations of the invention comprise processed Morinda citrifolia products. In one embodiment, the formulations include one or more extracts from the Morinda citrifolia L. plant. The Morinda citrifolia extracts preferably include Morinda citrifolia fruit juice, which juice is preferably present in an amount capable of maximizing prevention and/or treatment of substance abuse, addiction, withdrawal symptoms, anticipation associated with substance abuse in living organisms and associated symptoms. In another preferred embodiment the processed Morinda citrifolia product is administer in an amount sufficient to regulate dopamine levels, inhibit opioid receptors, inhibit mu opioid receptors, inhibit kappa opioid receptors, inhibit delta opioid receptors, act as NMDA antagonist, inhibit sensitization of NMDA receptors, and/or act as opiate antagonists without causing negative side effects when the composition is administered to a mammal.

### 4. EXAMPLES

Studies were conducted, which demonstrate that administration of the nutraceutical disclosed herein inhibit opiate receptors utilizing Morinda citrifolia L.

### Example 1

A study was conducted to evaluate in radioligand binding assays the activity of varying concentrations of processed Morinda citrifolia products. The methods employed in this study were adapted from scientific literature to maximize reliability and reproducibility. Reference standards were run as an integral part of each assay to insure the validity of the results obtained. The IC₅₀ values were determined by non linear, leased square regression analysis DATA ANALYSIS TOOLBOX™ (MDL Information Systems, San Leandro, Calif., USA). K₁ values were calculated using the equation of Cheng and Prusofs (Cheng, Y, Prusofs, W. H., Biochem. Pharacol. 22: 3099-3108, 1973) using the observed IC₅₀ of the tested compound concentration of radioligand employed in this assay, and the historical values for the K_{d} of the radioligand binding assays. The Hill coefficient (nₕ) defined the slope of the competitive binding curve was calculated using DATA ANALYAIS TOOLBOX™. Significant results are displayed in the following tables:

TABLE 1
PRIMARY TESTS

| PRIMARY | | | |
|---|---|---|---|
| BIOCHEMICAL ASSAY | SPECIES | CONC. | % INH. |
| Opiate δ (OP1, DOP) | hum | 1% | 77 |
| Opiate κ (OP2, KOP) | hum | 1% | 100 |
| Opiate μ (OP3, MOP) | hum | 1% | 93 |

Table 1 indicates that a 1% concentration of processed Morinda citrifolia product produced a 77% inhibition of the opiate Delta receptor. Further binding assays indicate that a 1% concentration of processed Morinda citrifolia product processed according to the methods of the present invention produced a 100% inhibition of opiate Kappa receptor and 93% inhibition of the opiate MU receptor.

TABLE 2
EXPERIMENTAL RESULTS-BIOCHEMICAL ASSAYS

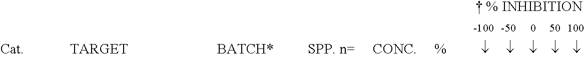

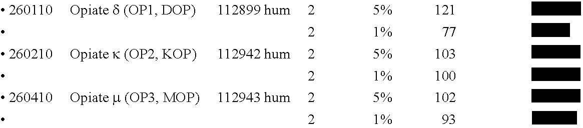

Table 2 demonstrates that in some cases increased concentrations of processed Morinda citrifolia product increased the percent inhibition of the opiate receptors and demonstrate the fact that at all tested concentrations the opiate receptors were significantly inhibited by processed Morinda citrifolia products. Table 2 demonstrates that 1% concentration Morinda citrifolia produced a 77% inhibition of the opiate Delta receptor, a 100% inhibition of the opiate Kappa receptor and a 93% inhibition of the opiate Mu receptor. Table 2 further demonstrates that a five percent concentration of processed Morinda citrifolia product produced a 121% inhibition of the opiate Delta receptor, 103% inhibition of the opiate Kappa receptor and a 102% inhibition of the opiate Mu receptor.

TABLE 3
METHODS - RADIOLIGAND BINDING ASSAYS

| 260110 Opiate δ (OP1, DOP) | |
|---|---|
| Source: | Human recombinant CHO cells |
| Ligand: | 0.9 nM [³H] Naltrindole |
| Vehicle: | 1% DMSO |
| Incubation Time/Temp: | 2 hours @ 25° C. |
| Incubation Buffer: | 50 mM Tris-HCl, 5 mM MgCl₂, pH |
| | 7.4 |
| Non-Specific Ligand: | 10 μM Naloxane |
| K_{D}: | 0.49 nM* |
| Bₘₐₓ: | 8.6 pmole/mg Protein* |
| Specific Binding: | 80%* |
| Quantitation Method: | Radioligand Binding |
| Significance Criteria: | ≧50% of max stimulation or |
| | inhibition |
| 260410 Opiate μ(OP3, MOP) | |
| Source: | Human recombinant CHO cells |
| Ligand: | 0.6 nM [³H] Diprenorphine |
| Vehicle: | 1% DMSO |
| Incubation Time/Temp: | 60 minutes @ 25° C. |
| Incubation Buffer: | 30 mM Tris-HCl, pH 7.4 |
| Non-Specific Ligand: | 10 μM Naloxane |
| K_{D}: | 0.41 nM* |
| Bₘₐₓ: | 3.8 pmole/mg Protein* |
| Specific Method: | 90%* |
| Quantitation Method: | Radioligand Binding |
| Significance Criteria: | ≧50% of max stimulation or |
| | inhibition |
| 260210 Opiate κ (OP2, KOP) | |
| Source: | Human recombinant HEK-293 cells |
| Ligand: | 0.6 nM [³H] Diprenorphine |
| Vehicle: | 1% DMSO |
| Incubution Time/Temp: | 60 minutes @ 25° C. |
| Incubation Buffer: | 50 mM Tris-HCl pH 7.4 |
| Non-Specific Ligand: | 10 μM Naloxane |
| K_{D}: | 0.4 nM* |
| Bₘₐₓ: | 1.1 pmole/mg Protein* |
| Specific Binding: | 90%* |
| Quantitation Method: | Radioligard Binding |
| Significance Criteria: | ≧50% of max stimulation or |
| | inhibition |

Table 3 indicates the source ligand incubation time and temperature incubation buffer, nonspecific ligand, the case of D, Bₘₐₓ, Specific Binding, Quantitation Method, and Significance Criteria for each of the receptor assayed in the radioligand binding assays of the research described.

TABLE 4
REFERENCE COMPOUND DATA - BIOCHEMICAL ASSAYS

| | | REFERENCE | HISTORICAL | | | CONCURRENT MIC | |
|---|---|---|---|---|---|---|---|
| CAT.# | ASSAY NAME | COMPOUND | IC₅₀ | K₁ | D_{H} | BATCH* | IC₅₀ |
| 260110 | Opiate δ (OP1, DOP) | Naltrindole | 0.92 nM | 0.32 nM | 1 | 112899 | 2.38 nM |
| 260210 | Opiate κ (OP2, KOP) | U-69593 | 0.016 μM | 6.4 nM | 0.5 | 112942 | 0.031 μM |
| 260410 | Opiate μ (OP3, MOP) | DAMGO | 0.02 μM | 8.1 nM | 0.6 | 112943 | 0.012 μM |

Table 4 provides the reference compound data utilized in the assays.

What is claimed is:

1.-12. (canceled)

13. A method for preventing substance abuse in mammals in mammals comprising the step of:
administering a formulation containing at least one processed Morinda citrifolia product present in an amount by weight between about 0.1 and 99 percent.

14. The method of claim 13, wherein two ounces of the formulation is administered twice daily.

15. The method of claim 13, wherein said Morinda citrifolia product is administered with a carrier medium.

16. The method of claim 13, wherein said processed Morinda citrifolia product comprises a processed Morinda citrifolia selected from a group consisting of: extract from the leaves of Morinda citrifolia, leaf hot water extract present in an amount by weight between about 0.1 and 50 percent, processed Morinda citrifolia leaf ethanol extract present in an amount by weight between about 0.1 and 50 percent, processed Morinda citrifolia leaf steam distillation extract present in an amount by weight between about 0.1 and 50 percent, Morinda citrifolia fruit juice, Morinda citrifolia extract, Morinda citrifolia dietary fiber, Morinda citrifolia puree juice, Morinda citrifolia puree, Morinda citrifolia fruit juice concentrate, Morinda citrifolia puree juice concentrate, freeze concentrated Morinda citrifolia fruit juice, and evaporated concentration of Morinda citrifolia fruit juice.

17. The method of claim 13, wherein the formulation comprises at least one active ingredient selected from a group consisting of quercetin, rutin, scopoletin, octoanoic acid, potassium, vitamin C, terpenoids, alkaloids, anthraquinones, nordamnacanthal, morindone, rubiandin, B-sitosterol, carotene, vitamin A, flavone glycosides, linoleic acid, Alizarin, amino acids, acubin, L-asperuloside, caproic acid, caprylic acid, ursolic acid, and putative proxeronines.

18. The method of claim 13, wherein the formulation further comprising at least one other ingredient selected from the group consisting of processed Morinda citrifolia products, food supplements, dietary supplements, other fruit juices, other natural ingredients, natural flavorings, artificial flavorings, natural sweeteners, artificial sweeteners, natural coloring, and artificial coloring.

19. The method of claim 13, further comprising the step of concurrently administering said formulation with another medication designed to improve lipoprotein profiles and its associated conditions, wherein said formulation increases the efficacy of said medication.

20. The method of claim 13, wherein said formulation is administered in an amount between about 1 teaspoon and 2 ounces at least twice daily on an empty stomach each day.

21. A method of treating mammals comprising:
administering a formulation containing at least one processed Morinda citrifolia product present in an amount by weight between about 0.1 and 99 percent, wherein the formulation is adapted to affect mammals in a way selected from a group consisting of: prevent addiction, treat withdrawal symptoms associated with substance abuse, prevent anticipation associated with substance abuse, act as a opioid antagonist, regulate dopamine levels, and act as a NMDA antagonist.
